(19) United States
(12) Patent Application Publication
(10) Pub. No.: US 2012/0282215 A1
(43) Pub. Date: Nov. 8, 2012

(54) Vaccination of Cancer Patients

(75) Inventors: Jonathan F. Head, Baton Rouge, LA (US); Robert L. Elliott, Baton Rouge, LA (US)

(73) Assignee: OncBioMune, L.L.C., Baton Rouge, LA (US)

(21) Appl. No.: 13/005,984

(22) Filed: Jan. 13, 2011

(51) Int. Cl. A61K 38/20; A61P 35/00; A61P 35/02; A61P 37/04

(52) U.S. Cl. 424/85.2

(57) ABSTRACT

Pharmaceutical compositions containing (i) whole autologous cancer cells, whole allogeneic cancer cells, or a combination thereof; (ii) a colony stimulating factor (CSF); (iii) an interleukin; and (iv) a pharmaceutically acceptable carrier are described which are useful for inhibiting tumor growth, progression or recurrence or to inhibit metastases formation in solid cancerous tumors, leukemias, and lymphomas.

### FIELD OF THE INVENTION

The present invention is directed to compositions useful as vaccines for cancer patients.

### BACKGROUND OF THE INVENTION

There has been considerable effort by researchers to develop compositions useful for vaccinating cancer patients. The goal of such therapeutic vaccines is to stimulate the immune system of patients to attack existing cancer cells. To accomplish this, attempts have been made to vaccinate such patients with tumor cells or tumor-associated antigens. Many of these efforts have had limited success.

Previous studies with lymphokine/cytokine agents, including interleukin-2 (IL-2) and granulocyte macrophage colony stimulating factor (GM-CSF), to treat patients with various types of cancer, have demonstrated, at best, weakly positive results, in part because varying degrees of toxicity had been associated with the use of high doses of both adjuvants.

One example of a class of vaccines is reported in U.S. Pat. No. 5,478,556, which describes vaccinating breast cancer patients with compositions comprising a combination of extracted autologous breast tumor associated antigens or allogeneic breast tumor associated antigens obtained from cancer cells, interleukin-2 (IL-2), and granulocyte macrophage colony stimulating factor (GM-CSF).

### SUMMARY OF THE INVENTION

In a first aspect, pharmaceutical compositions are provided comprising (i) whole autologous cancer cells, whole allogeneic cancer cells, or a combination thereof; (ii) a colony stimulating factor (CSF); (iii) an interleukin; and (iv) a pharmaceutically acceptable carrier.

In a second aspect, pharmaceutical compositions are provided comprising, in a liquid, pharmacologically acceptable carrier, about 100,000 to about 10 million autologous breast cancer cells, or about 100,000 to about 10 million allogeneic breast cancer cells, or a combination thereof, about 5,000 to about 50,000 Units of an interleukin, and about 10 to about 100 μg of a colony stimulating factor (CSF), per 100 μL of composition.

In another aspect, methods for treating a cancer in a patient are provided comprising, administering to a human patient in need thereof a therapeutically effective amount of a composition of the first or second aspects, wherein a human patient has a solid cancerous tumor, a leukemia, or a lymphoma.

In another aspect, methods of enhancing the immune response in a human patient who has a solid cancerous tumor, leukemia, or lymphoma to inhibit tumor growth or recurrence or to inhibit metastases formation are provided comprising administering to the patient a therapeutically effective amount of a composition of the first or second aspects.

The administration of such compositions to cancer patients has been found to elicit immunological responses and clinical responses in the patients. Using whole cells as the basis for the vaccine confers certain advantages including the likelihood that the cells used have most or all of the tumor antigens of the tumor type from which they are derived. Furthermore, it is likely that these tumor antigens on autologous or allogeneic cells are expressed preferentially over antigens expressed on whole cells derived from normal tissue.

### BRIEF DESCRIPTION OF THE FIGURES

FIG. 1 is a graph showing the ratio (after vaccination:before vaccination) of lymphocyte blastogenesis assay (LBA) proliferation index values (stimulation index) obtained from 41 patients for each component of the vaccine.

FIG. 2 is a graph showing the overall survival of breast cancer patients after vaccination with a breast cancer vaccine containing whole cells and tumor marker proteins, and the overall survival of historic controls of breast cancer patients with depressed and normal immunity.

### DETAILED DESCRIPTION OF THE INVENTION

As noted above, it is known that tumor associated antigens hypotonically extracted from solid tumor cells can be combined with, for example, IL-2 and GM-CSF and be useful as cancer vaccines. It now has been discovered that the extraction procedure is not required and that whole autologous or allogeneic cancer cells from any of a variety of solid tumor cancers, administered in combination with low doses of an interleukin and a CSF, such as IL-2 and GM-CSF, respectively, are useful as vaccines.

Compositions comprising autologous or allogeneic cancer cells, or a combination of both autologous and allogeneic cancer cells, have found to be useful as cancer vaccines when administered in combination with low doses of an interleukin and a CSF.

Such compositions further can comprise one or more tumor marker proteins associated with the type of tumor to be treated.

The compositions can be administered to patients suffering from solid tumors, leukemias or lymphomas to obtain an immunotherapeutic reaction which can result in the inhibition of tumor growth or recurrence or to inhibit formation of metastases.

Such vaccines are prepared by first obtaining a sample of tissue from the type of tumor to be treated and digesting it in accordance with conventional procedures. In one embodiment, whole allogeneic cancer cells can be obtained by: (i) digesting a tumor tissue to recover viable and intact tumor cells; or (ii) collecting cells from tumor cell lines; or (iii) separating tumor cells from blood to recover viable and intact tumor cells; and treating the viable and intact tumor cells to yield non-viable tumor cells. In another embodiment, whole autologous cancer cells can be obtained by: (i) digesting a tumor tissue to recover viable and intact tumor cells; or (ii) collecting cells from tumor cell lines; or (iii) separating tumor cells from blood to recover viable and intact tumor cells; and treating the viable and intact tumor cells to yield non-viable tumor cells.

As noted above, the tumor tissue can be from the patient's own tumor and/or from tumor tissue obtained from one or more other persons diagnosed with cancer of the same organ as the patient of interest. A suspension is prepared containing the digested tissue and centrifuged. The resulting supernatant is discarded and the pellet of tumor cells is collected. If the cell pellet contains sufficient cells for a vaccine, the cells are rendered non-viable, for example, by treatment with Mitomycin C or irradiation in accordance with known procedures. The cells can then be resuspended in a salt solution.

The vaccine can be prepared and used promptly as described below, or, if the vaccine will not be used promptly, the cells that are suspended in the salt solution can be aliquoted into freezer vials and frozen. Then, at an appropriate time, the cells can be thawed and used as the basis for the vaccine. Generally, as described below, about 100,000 cells to about 10 million cells; or about 100,000 cells to about 5 million cells; or about 500,000 to 5 million, or about 500,000 to about 1 million cells, of each cell type, independently, can be used as the basis of a vaccine composition. The term “about” as used herein means +/−10% of the referenced value.

If the cell pellet does not contain sufficient cells for a vaccine (or if cells in excess of those needed for a vaccine are desired for any other purpose), the cell pellet can be re-suspended in an appropriate cell culture medium and selectively expanded. If the cell expansion is not to be carried out promptly, the cells can be re-suspended and frozen until needed as described above. Procedures and growth media for growing such cells are described in U.S. patent application Ser. No. 10/918,741, filed Aug. 16, 2004, now U.S. Patent Application Pub. No. US 2006/0035375, which is incorporated herein by reference. Specifically, the cell pellet initially is suspended in a first growth medium comprising D-valine MEM, methyl cellulose, serum, glutamine and an antibiotic, wherein the methyl cellulose component of the medium is present at a concentration sufficient to inhibit the growth of fibroblast cells present in the cell pellet. The suspension is added to a cell culture vessel, the inner surface of which has been at least partially coated with a medium comprising a protein extract, D-valine MEM, glutamine and an antibiotic, then the suspension is incubated in the vessel to allow selective growth of the epithelial cells or carcinoma cells. The cells then are rinsed with a balanced salt solution, and the first growth medium can be replaced with a second growth medium comprising D-valine MEM, serum, glutamine and an antibiotic but essentially free of methyl cellulose. The desired epithelial or cancer cells subsequently are recovered from the second growth medium.

Vaccine compositions can be prepared comprising the expanded autologous and/or allogeneic cells. Desirably, each dose of vaccine comprises about 100,000 cells to about 10 million of each cancer cell type, independently, or about 100,000 to about 5 million; or about 500,000 to about 5 million cells, or about 500,000 to 1 million cells, per 100 μL of vaccine.

In one embodiment, a vaccine comprises about 1 million autologous cells and about 1 million allogeneic cells.

The cells are combined in the vaccine composition with small amounts of a combination of an interleukin and CSF.

Suitable interleukins include IL-1, IL-2, IL-3, IL-4, IL-5, IL-6, IL-7, IL-9, IL-12, IL-13, IL-14 and IL-15. In certain embodiments, the interleukin is IL-1. In certain other embodiments, the interleukin is IL-2. In certain other embodiments, the interleukin is IL-3. In certain other embodiments, the interleukin is IL-4. In certain other embodiments, the interleukin is IL-5. In certain other embodiments, the interleukin is IL-6. In certain other embodiments, the interleukin is IL-7. In certain other embodiments, the interleukin is IL-9. In certain other embodiments, the interleukin is IL-12. In certain other embodiments, the interleukin is IL-13. In certain other embodiments, the interleukin is IL-14. In certain other embodiments, the interleukin is IL-15.

Suitable colony stimulating factors include granulocyte-macrophage-CSF (GM-CSF), granulocyte-CSF (G-CSF) and macrophage-CSF (M-CSF). In certain embodiments, the colony stimulating factor is GM-CSF. In certain embodiments, the colony stimulating factor is G-CSF. In certain embodiments, the colony stimulating factor is M-CSF.

Although the effectiveness, measured in terms of immune response and overall survival, of each type of cytokine is very limited if administered alone, the administration of both an interleukin and a CSF in combination with the autologous and/or allogeneic cancer cells has been found to have a synergistic effect. Specifically, administering both an interleukin and a CSF in combination improves immune response and overall survival, where administration of each cytokine separately does not.

In one embodiment the combination of cytokines is GM-CSF and IL-2. The IL-2 promotes cytotoxic T-cell immunity; the GM-CSF promotes dendritic cell processing.

Typically, the composition comprises from about 5,000 Units (0.30 μg) to about 50,000 Units (U) (3.05 μg) of the interleukin per 100 μL of vaccine, or from about 10,000 U (0.61 μg) to about 30,000 U (1.83 μg) of the interleukin per 100 μL of vaccine, or about 20,000 U (1.22 μg) of the interleukin per 100 μL of vaccine; and about 10 μg to about 100 μg of the CSF per 100 μL of vaccine, or about 15 μg to about 20 μg of the CSF per 100 μL of vaccine, or about 16.5 μg to about 16.9 μg of the CSF per 100 μL of vaccine, or about 16.7 μg of the CSF per 100 μL of vaccine.

In another embodiment, the vaccine composition further comprises one or more tumor marker proteins. In one embodiment, the compositions further comprise at least one tumor marker protein associated with the type of tumor to be treated.

Tumor marker proteins can be selected based upon the type of tumor to be treated. For example, if the cancer to be treated is breast cancer, suitable tumor marker proteins can include carcinoembryonic antigen (CEA), CA 125 protein and CA 15-3 protein.

If the cancer to be treated is liver cancer, suitable tumor marker proteins can include alpha feto-protein (AFP). If the cancer to be treated is prostate cancer, a suitable tumor marker protein is prostate specific antigen (PSA).

A marker for colorectal cancer is carcinoembryonic antigen (CEA); this antigen also can serve as a marker for other cancers such as pancreas, stomach, breast, lung and thyroid cancers.

A recognized marker of germ cell tumors, such as testicular or ovarian cancer, is beta HCG. CA 19-9 is a recognized tumor marker for adenocarcinomas such as digestive tract cancers or intra-abdominal carcinomas; CA 125 is a recognized marker for ovarian cancer as well as breast cancer; and AFP is a marker for ovarian, stomach and pancreas tumors, as well as for liver tumors.

Squamous cell carcinoma antigen (SCC antigen) is a marker for squamous cell cancers, which can occur in the cervix, head, neck, lung and skin. Vaccine compositions can also be made with one or more of the tumor marker proteins identified in Table 1.

TABLE 1

| Cancer | Associated Tumor Markers |
|---|---|
| breast | CA 15-3, CA 27.29, CEA, CA 125, ER, PgR, |
| | Her-2/neu, LASA-P |
| prostate | PSA, PAP, PSMA, CEA, CA 125 |
| gastrointestinal | CEA, CA 19-9, AFP, CA 72-4, BTA, TPA |
| lung | CEA, AFP, NSE, Chromogranin A, TPA, CA 72-4 |
| liver | CEA, CA 19-9, AFP |
| pancreatic | CEA, CA 19-9, AFP, CA 50 |
| thyroid | CEA, Calcitonin, Thyroglobulin |
| ovarian | CA 125, AFP, LASA-P, CA 50, CA 72-4 |
| melanoma (skin) | S-100, TA-90 |
| testicular | AFP, β-hCG |
| leukemia | β-2 microglobulin, LASA-P |
| colorectal | CEA, CA 19-9, LASA-P |
| bladder | BTA, NMP 22, TPA |
| lymphoma | LASA-P |
| endometrial | ER, PgR |
| nervous system | Calcitonin, Chromogranin A, NSE |
| embryonic | β-hCG |

Persons of skill in the art can identify tumor marker proteins which are associated with a cancer of interest. Any surface or secreted protein which can be over-expressed by the type of tumor to be treated can be included in the vaccine composition. The tumor marker protein(s) are each independently present in amounts ranging from about 1 μg to about 100 μg per 100 μL of vaccine composition.

For example, if the cancer to be treated is breast cancer and CEA antigens are included in the vaccine they are provided at a concentration of about 1 μg to about 10 μg, or about 2 μg, per 100 μL; if CA 125 protein is included it can be provided at a concentration of from about 100 IU to about 10,000 IU, or about 1000 IU per 100 μL; if CA 15-3 is included it can be provided at a concentration of about 1 μg to about 100 μg per 100 μL.

In another example, if the type of cancer of interest is prostate cancer and PSA is included in the vaccine composition. PSA can be provided at a concentration of about 5 μg to about 500 μg, or about 50 μg per 100 μL. Other tumor marker proteins can be provided at a concentration of about 1 μg to about 100 μg per 100 μL of vaccine composition, or about 50 μg per 100 μL of vaccine composition.

These amounts of tumor marker proteins are used in each vaccine, whether they are used individually or in combination with other tumor marker proteins.

Suitable pharmaceutically acceptable carriers for the compositions refers to a fluid vehicle that can be injected into a host without significant adverse effects. Suitable pharmaceutically acceptable carriers known in the art include, but are not limited to, sterile water, saline, glucose solution, and physiologically acceptable aqueous buffers or solutions, including phosphate-buffered saline. Carriers may include auxiliary agents including, but not limited to, diluents, stabilizers (i.e., sugars and amino acids), preservatives, wetting agents, emulsifying agents, pH buffering agents, viscosity enhancing additives, colors and the like.

Appropriate amounts of the proteins and cells are mixed with the selected carrier to form the final vaccine composition. In one embodiment, all the components of the vaccine can be provided together in one carrier, but if desired, one or more components can be provided in a separate carrier and administered in combination with the other components.

The vaccine can be administered by injection. In another embodiment, the vaccine is administered subcuticularly, but it also can be administered subcutaneously, intradermally, intramuscularly, intravenously or intraperitoneally. The vaccine typically is administered in doses of about 0.3 mL to about 1 mL, or 0.5 mL to about 1 mL, or about 0.7 mL per dose.

Although a single dose of the vaccine may be useful, oftentimes it is desirable to administer from about 1 to about 12 doses of the vaccine. If more than one dose is administered, the doses typically are administered at least about 7 days apart. For example, the doses typically can be administered at about 7 to about 90 days apart. For example, one useful regimen is to administer three doses of the vaccine at weekly intervals, then to administer a further three doses at four week intervals after that (i.e., the vaccine is administered at weeks 0, 1, 2, 6, 10 and 14).

The vaccine can be administered alone or in conjunction with chemotherapy, depending on the stage and grade of the cancer and the patient's overall health status. In one embodiment, the vaccine is given in combination with chemotherapy to a patient with metastatic cancer. In another embodiment, the administration of vaccine can be alternated with the administration of IL-2.

The T- and B-cell immunity of a patient receiving a vaccine in accordance with this disclosure can be monitored using a lymphocyte blastogenesis assay before beginning the vaccine administration and then during and after the vaccine regimen. (The assay is described in Head, J. F., Elliott, R. L., and McCoy, J. L.: Evaluation of Lymphocyte Immunity in Breast Cancer Patients. Breast Cancer Res. Treat. 26:77-88, 1993); and Head, J. F., Wang, F. Elliott, R. L., and McCoy, J. L.: Assessment of Immunological Competence and Host Reactivity Against Tumor Antigens in Breast Cancer Patients: Prognostic Value and Rationale of Immunotherapy Development. Ann. New York Acad. Sci. 690:340-342, 1993. These references are herein incorporated by reference.) In one embodiment, immunity is tested after 3, 6 or 12 vaccinations. An increase in the stimulation index demonstrates an increase in the immune response to the cellular and protein antigens by the vaccination process. Other methods of monitoring the patient's immune status comprise measuring serum tumor markers and the level of circulating IL-6.

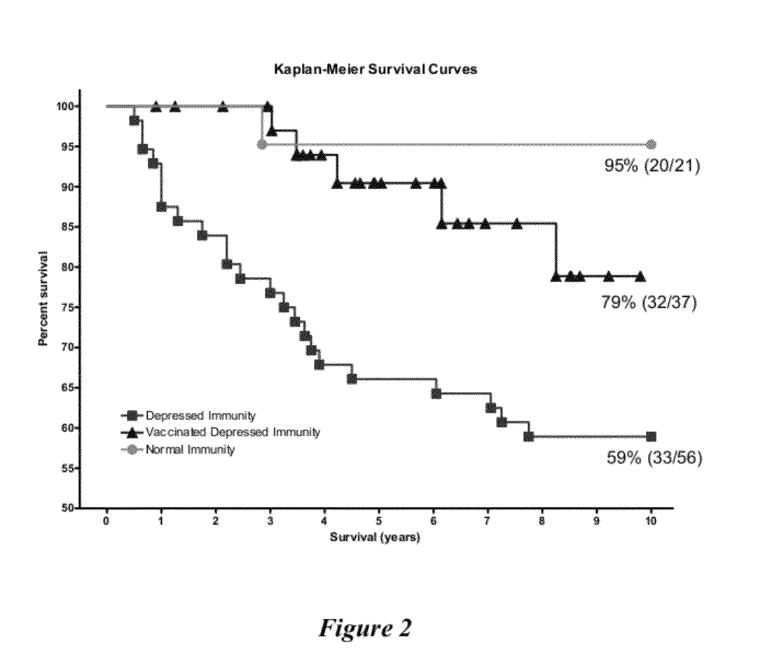

In the group of patients in FIG. 2 with depressed immunity who were vaccinated with a therapeutic breast cancer vaccine containing whole cells and tumor marker proteins, there was an improvement in their immune responses and also, as shown in FIG. 2, a 34% improvement in overall survival.

Vaccines in accordance with this disclosure are useful for the treatment of any solid tumor cancer, including cancers of the breast, lung, colon, rectum, prostate, uterus (including cervix and endometrium), ovary, oral cavity, bladder, pancreas, stomach, kidney, skin, testicles and lymphoid tissue. The vaccines also can be used to treat leukemia. Appropriate autologous or allogeneic cells are obtained, and, if desired, appropriate tumor marker proteins are selected. In one embodiment, the donor materials are a cell line growing in continuous cell culture. In another embodiment, the donor tissue is of the same tumor type and cell type as the patient's tumor. In another embodiment, the donor tissue is matched to the recipient's HLA type.

The vaccine is prepared using the general amounts of the components of the vaccine as set forth above.

As used herein, the phrase “therapeutically effective amount” refers to the amount of referenced compound or composition that elicits the biological or medicinal response that is being sought in a tissue, system, animal, individual or human by a researcher, veterinarian, medical doctor or other clinician, which includes one or more of the following:
(1) preventing the disease; for example, preventing a disease, condition or disorder in an individual who may be predisposed to the disease, condition or disorder but does not yet experience or display the pathology or symptomatology of the disease;
(2) inhibiting the disease; for example, inhibiting a disease, condition or disorder in an individual who is experiencing or displaying the pathology or symptomatology of the disease, condition or disorder; and
(3) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.
   For example, a therapeutically effective amount of a compound or composition can be measured as an amount that increases the immune response to tumor antigens by at least 50%; or stabilizes or decreases circulating tumor marker proteins resulting in inhibiting the progression of the cancer.

As used here, the terms “treatment” and “treating” means (i) ameliorating, inhibiting, or preventing the referenced disease state, as described above; or (ii) eliciting the referenced biological effect (e.g., an immunotherapeutic reaction can result in an alleviation or arrest of the disease from which the patient of interest is suffering). More specifically, treating can include inhibiting tumor growth or recurrence, regressing a tumor and/or inhibiting formation of metastases.

The preceding is further illustrated by the following examples, which are not intended to be limiting.

### EXAMPLES

### Example 1

### Preparation of Vaccine

A composition to be used as a vaccine for a breast cancer patient was prepared containing the following components:

TABLE 2

| Component | Quantity | | Volume |
|---|---|---|---|
| Autologous cells | 1 × 10⁶ | cells | 100 μL |
| MCF-7 cells | 1 × 10⁶ | cells | 100 μL |
| CA 15-3 protein | 50 | μg | 100 μL |
| CEA protein | 2 | μg | 100 μL |
| CA 125 protein | 1000 | IU | 100 μL |
| IL-2 | 20,000 | U | 100 μL |
| GM-CSF | 16.7 | μg | 100 μL |

| | |
|---|---|
| Total Volume | 700 μL |

### Autologous Whole Cell Preparation

A human autologous whole cell preparation for a vaccine begins with a sample of tumor tissue from a human cancer patient. The tissue sample was digested in accordance with conventional procedures. Typically, small pieces of tissue in a sterile transport medium were digested with enzymes such as collagenase and DNase.

A piece of breast tumor tissue (1 cm³ or approximately 1 gram) obtained from a female breast cancer patient at biopsy was cut into 3 pieces. The pieces were chilled in 15 mL of sterile transport medium (alpha-MEM, 10% fetal calf serum, 2 mM L-glutamine, 50 mg/Liter gentamicin) at 4° C. Upon arrival at the laboratory, the chilled tissue and transport medium were transferred into a petri dish and the tissue cut into small pieces (about 1 mm cubes) in the chilled transport medium.

The chilled medium containing the chilled small pieces of breast tumor tissue was poured into a spinner flask. 4.5 mL of 3% collagenase (Collagenase, type 3, Product #CLS-3, Worthington Biochemical, Lakewood, N.J.) and 4.5 mL of 0.02% DNase (Deoxyribonuclease I, Type IV, Product #D 5025, Sigma, St. Louis, Mo.) were added to the medium. The resulting enzyme digest was incubated with spinning for 5 hours at 37° C. The incubated digest was poured into a 50 mL conical centrifuge tube and brought to 40 mL with Hanks Balanced Salt Solution (HBSS) without calcium or magnesium (Product #H 6648, Sigma, St. Louis, Mo.). The number of cells in the 40 mL cell suspension was counted with a hemocytometer (seeking about 10,000,000 cells for the vaccine). The 40 ml, cell suspension was centrifuged at 1000 g for 15 minutes. The resulting supernatant was discarded and the pellet of breast tumor cells was collected.

The cells from primary digests were rendered non-viable by Mitomycin C treatment or irradiation. The cell pellet was resuspended in 200 μl of HBSS, 100 μl of Mitomycin C (150 μg/mL) was added to the tube and incubated at room temperature for 30 minutes. The cells were washed with 10 mL of HBSS and centrifuged at 100 g for 15 minutes. The cell pellet was resuspended at a concentration of 10 million cells/mL in HBSS with 10% DMSO and aliquoted into 0.1 mL quantities, containing 1.0 million cells, into small freezer vials and stored at −80° C. for future use. This was as described in the patent application U.S. Ser. No. 10/918,741, METHOD FOR SELECTIVELY CULTURING EPITHELIAL OR CARCINOMA CELLS, Inventors: Head et al.; herein incorporated by reference.

### Cell Culture Cell Preparation

MCF-7 cells or any cancer cell grown in culture can be used as a source of antigens for vaccines to induce cellular and humoral immune responses. MCF-7 breast carcinoma cells were obtained from ATCC and grown in alpha-MEM containing 10% fetal calf serum, 2 mM L-glutamine and 50 mg/Liter gentamicin. When the cell density was approaching confluence the cells in the T-75 cell culture flask were rinsed twice with sterile HBSS. The attached cells were removed from the plastic surface of the T-flask by digestion with 3 mL Trypsin-EDTA solution (0.25% trypsin and 0.02% EDTA) for 5-10 minutes. When the cells detach, enzyme activity was inhibited by adding 7 mL of complete alpha-MEM. The detached cells in media were transferred into a 50 mL conical centrifuge tube and brought to 40 mL with Hanks Balanced Salt Solution (HBSS) without calcium or magnesium (Product #H 6648, Sigma, St. Louis, Mo.). The number of the cells in the 40 mL cell suspension was counted with a hemocytometer. The 40 mL cell suspension was centrifuged at 1000 g for 15 minutes. The resulting supernatant was discarded and the pellet of tumor cells was collected.

The cells were rendered non-viable by Mitomycin C treatment or irradiation. The cell pellet was resuspended in 200 μL of HBSS, 100 μl of Mitomycin C (150 μg/ml) was added to the tube and incubated at room temperature for 30 minutes. The cells were washed with 10 mL of HBSS and centrifuged at 1000 g for 15 minutes. The cell pellet was resuspended at a concentration of 10 million cells/ml in HBSS with 10% DMSO and aliquoted into 0.1 mL quantities, containing 1.0 million cells, into small freezer vials and stored at −80° C. for future use.

### Tumor Antigen Preparation

CA 15-3 (Fitzgerald Industries International, Inc., Concord, Mass.). Final concentration 50 μg in 100 μl sterile water, USP.

CEA (Fitzgerald Industries International, Inc., Concord, Mass.) Final concentration 2 μg in 100 μl sterile water, USP.

CA125 (Fitzgerald Industries International, Inc., Concord, Mass.) Final concentration 1000 IU in 100 μl sterile water, USP.

### Adjuvant Preparation

IL-2 (Proleukin, Chiron Corporation, Emeryville, Calif.) Final concentration 20,000 IU in 100 μl. Supplied as lyophilized powder, 22×10⁶ IU (1.3 mg)/vial. Added 1.1 mL of sterile water, USP. Serially to dilute 1:10, 1:10 (final 1:100). Final concentration 20,000 IU (1.18 μg) in 100 μL.

GM-CSF (Leukine, Immunex Corp., Seattle, Wash.). Supplied as a lyophilized powder 250 μg/vial. Added 1.5 mL of sterile water, USP. Final concentration 16.7 μg in 100 μL. Store at 4° C. and use 100 μL in vaccine.

### Example 2

### Response to Cell and Tumor Antigens

A trial of the vaccine was begun by vaccinating 41 breast cancer patients. Patients were selected who had depressed T- and B-cell immunity, as determined in a Lymphocyte Blastogenesis Assay (LBA), and the patients' immune responses to the vaccine were also monitored with the LBA. Fifteen patients had autologous cells in their vaccines, 41 patients had allogeneic cells (MCF-7 cells) in their vaccine, 39 patients had cancer antigen 15-3 in their vaccines, 39 had cancer antigen CEA in their vaccines and 37 patients had cancer antigen 125 in their vaccines.

TABLE 3

| | NUMBER |
|---|---|
| COMPONENTS | OF PATIENTS |
| autologous, allogeneic, CA 15-3, CEA, CA-125 | 12 |
| allogeneic, CA 15-3, CEA, CA-125 | 25 |
| autologous, allogeneic, CA 15-3, CEA | 2 |
| autologous, allogeneic | 1 |
| allogeneic | 1 |

Sixty-eight percent (28 of 41) of the patients were greater than 50 years old. The vaccine (prepared according to Table 3) was injected subcuticularly in the skin of the femoral triangle (inner thigh about 6 inches below the inguinal lymph nodes) on weeks 0, 1, 2, 6, 10 and 14. Twenty-nine percent (12 of 41) of the patients had metastatic disease when the vaccination series was started, and therefore were Stage IV. Seventy-three percent (30 of 41) of patients had estrogen receptor positive tumors, and fifty-four percent (22 of 41) of the patients had progesterone receptor positive tumors. Seventy-eight percent (32 of 41) of the patients received chemotherapy before vaccine, 34% (14 of 41) received no chemotherapy or antihormone therapy during vaccination series, 34% (14 of 41) received chemotherapy during vaccination series, 29% (12 of 41) received antihormone therapy during the vaccination series, and one patient received both chemotherapy and antihormone therapy.

The Lymphocyte Blastogenesis Assays (LBA) was used to determine the ability of T- and B-lymphocytes to proliferate in response to tumor antigens and was done as we have previously reported (Jiang et al. Cancer Biother Radiopharm 15:110, 2000). Blood (40 mL) was collected in green top vacutainer tubes containing sodium heparin and divided into two portions of 20 mL each in 50-mL sterile tubes. The following materials and reagents were sterile. Ten milliliters of HBSS were pipetted into each portion of blood. Then, 9 mL of Sigma Histopaque-1077 (St Louis, Mo.), brought to room temperature, were carefully pipetted into the bottom of each tube. The Histopaque-1077 should remain below the blood without disturbing the blood-Histopaque discontinuous gradient. Tubes were centrifuged at 400×g for 30 minutes at room temperature. After centrifugation there were 3 distinct bands. Being careful not to disturb the contents of the tubes, the middle band (containing the lymphocytes) was pipetted into another tube. Lymphocytes were counted and centrifuged at 250×g for 10 minutes. The pellet of lymphocytes was resuspended in serum-free Sigma QBSF-56 media (St Louis, Mo) containing 4×10⁶ cells per mL of media. Cells were plated in 96-well plates (Becton Dickinson Labware, Franklin Lakes, N.J.) at 4×10⁵ cells per well (in 100 μL). The media control of 12 wells in the top row contained 100 microliters of working QBSF-56 media and 100 microliters of cells. Cells or tumor antigens were serially diluted yielding 1:10, 1:100, 1:1,000 and 1:10,000 dilutions. One hundred microliters of each dilution of each antigen was added to triplicate wells of the second through sixth row of 96-well plates. One hundred microliters of positive control, consisting of a serial dilution (1:10, 1:100, and 1:1,000) of stock phytohemagglutinin (PHA, 0.5 mg/mL) in QBSF-56, was placed into triplicate wells of the first 9 wells of the 8th row. One hundred microliters of negative control (1:1000 dilution of healthy male plasma in QBSF-56 media) were added to each of last 3 wells of the 8th row. The plate was incubated for 6 days and then pulsed with 50 microliters of ³H-thymidine for exactly 18 hours and harvested. The radioactivity (counts per minute, cpm) was counted by scintillation spectrometry. The cpm of PHA dilution with the highest cpm was divided by the average cpm of the media control. If this ratio was greater than 2.00, the positive control was accepted. The cpm of the negative control was divided by the average cpm of the media control. If this ratio was less than 2.00 then the negative control was accepted. The lymphocyte proliferation index was calculated as the ratio of cpm of antigen dilution with highest cpm to the average cpm of media control.

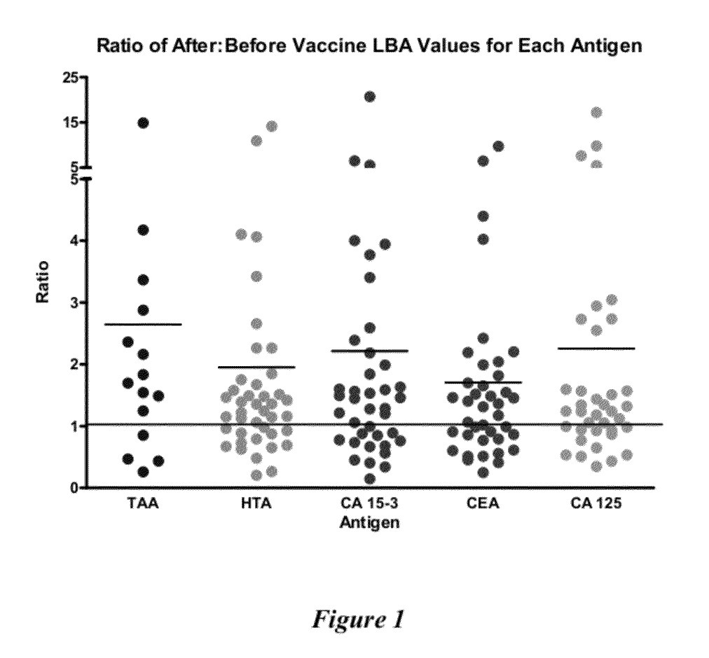

The lymphocyte blastogenesis assay was repeated for each patient four weeks after the sixth vaccine, and the stimulation index for each vaccine component for each patient was determined by dividing the proliferation index after vaccination by the proliferation index before vaccination. Any value above 1.00 constitutes an increased immune response. The results of this comparison were provided in FIG. 1 and clearly illustrate a definite increase in the immune response to the cellular and protein antigens by vaccination. The average stimulation index, which was the ratio of the after vaccine to before vaccine LBA results, was significantly over 1.00 for both cellular antigen preparations (autologous cells and allogeneic cells) and the three tumor marker protein antigens (CA 15-3, CEA and CA 125), as shown in FIG. 1. This demonstrates that the vaccine increased the host immune response to all five antigens found in the vaccine. Breast cancer patients who had a clinical response to the vaccine also showed a corresponding increase in cellular and/or humoral immunity.

The data in Table 4 below show the number of patients with a stimulation index greater than 1.5, a 50% increase in immune response, for the two cellular antigens (autologous cells and allogeneic cells, for example MCF-7 Cells), and the three tumor marker protein antigens in the LBA. A 50% increase in the stimulation index was a surrogate marker for an immune response. The data show that 60% of the patients responded to the autologous cellular antigens by an increase of at least 50% in their pre- to post-vaccine LBA proliferation index (stimulation index). Not unexpectedly, a smaller proportion of patients (32%) responded to the allogeneic cellular antigens. The tumor antigen from breast cancer (the CA 15-3 protein) had a 44% response rate (proportion of patients with at least a 50% increase in their stimulation index), and each of the CEA and CA 125 protein antigens had a slightly lower response rate, 35% and 36%, respectively.

TABLE 4

| Antigen | Response | % |
|---|---|---|
| Autologous cells | 9/15 | 60.0 |
| Allogeneic cells | 13/41 | 31.7 |
| CA 15-3 | 17/39 | 43.6 |
| CA 125 | 13/37 | 35.1 |
| CEA | 14/39 | 35.9 |

The number of patients treated with each antigen varied for two reasons. Only 15 patients had their fresh tumors processed for autologous cells. The other patients had no tissue processed for autologous cell vaccines because either the biopsy was done by another surgeon, or the tumor was so small that all the tumor was used for diagnosis and immunohistochemistry for therapeutic and prognostic indications. All patients did receive the allogeneic cells in their vaccines. CA 15-3 and CA 125 were added to the vaccine after the first two patients had already started their vaccine series and CEA was added to the vaccine after four patients had started their vaccine series. The vaccine caused an increase in the LBA response of 60% to autologous cells and only 32% to the allogeneic MCF-cells. This higher response rate to the autologous cells in the LBA was consistent with the autologous cells in the vaccine preparation having the same antigens on the surface as the patient's own tumor. This was in contrast to the allogeneic cell lines having either qualitatively different surface antigens and/or quantitatively less of a specific antigen. Further the allogeneic cells were not HLA matched to all the patients, so the immune systems of some patients were not able to process the antigens from the allogeneic cells and, therefore, were not able to produce an immune response.

TABLE 5

| # cell types or antigens | | |
|---|---|---|
| responded to | # Patients total | % Patient Responding |
| 0 | 10/41 | 24.4 |
| 1 | 13/41 | 31.7 |
| 2 | 6/41 | 14.6 |
| 3 | 8/41 | 19.5 |
| 4 | 3/41 | 7.3 |
| 5 | 1/41 | 2.4 |

Table 5, above, shows that, while 24% of the patients had no responses to any of the 5 cell types or antigens, 76% of the breast cancer patients had an increased immune response (ratio of after vaccine proliferation index to before vaccine proliferation index, previously defined as stimulation index, of greater than 1.5 was defined as an increase in response to cell types or protein antigens) to at least one of the cell types or protein antigens in the breast cancer vaccine. This stimulation index of 1.5 represents an immune response with a high probability of producing a significant clinical response. The patients in Table 5 were the same 41 patients from Table 4 and used the same LBA data as in Table 3. There was a steady decrease in percent of patients responding to increasing numbers of cell types or antigens. Thirteen of 41 patients (32% of patients) had an increased immune response to one cell type or antigen after vaccination. The percentage of patients having an increased immune response to cell types or antigens with increasing number of cell types or antigens steadily decreased until only one patient out of 41 (2.4% of patients) had an increased response to all cell types and antigens. The decreasing proportion of patients who responded to an increasing number of cell types or antigens suggests that the immune response to each cell type or antigen was independent of the response to any other and, therefore, that response to one cell type or antigen in any one patient does not make it more likely for that patient to have an immune response to another tumor cell type or antigen. However, the more cell types or antigens that were included in the vaccine the more patients who will have a response to at least one cell type or antigen. Also, patients with immune responses to multiple cell types or antigens were more likely to have a clinical response.

### Example 3

### Retrospective Study

We have done a retrospective study of our early stage breast cancer patients with depressed immunity that had the present breast cancer vaccine in the adjuvant setting. They were tested for immune responses to autologous cells, allogeneic cells and protein antigens. Patients with depressed immunity, a ratio less than 1.5 in the LBA, were vaccinated with a minimum of six vaccines, as described earlier.

The follow-up has been for up to 10 years. FIG. 2 is the Kaplan-Meier Overall Survival Curve with patients who died of other causes censured. The two control lines on the curve come from a previous study (Head et al. Assessment of Immunological Competence and Host Reactivity Against Tumor Antigens in Breast Cancer Patients: Prognostic Value and Rationale of Immunotherapy Development. Ann. New York Acad. Sci. 690:340-342, 1993) we published on the overall survival of patients with normal and depressed immunity with three patients dying from other causes being censured. That study showed that depressed immunity was related to poor prognosis, as demonstrated by a comparison of the two control lines (i.e., the depressed immunity and normal immunity groups) in FIG. 2. The calculated Kaplan Meier overall survival for the control groups at 10 years was 59% survival in depressed patients with standard treatment and 95% survival for patients with immunity to their tumor associated antigens in a LBA at presentation with standard treatment. Neither control group was vaccinated. However, the calculated Kaplan Meier overall survival for the vaccinated patients at 10 years was 79% with standard treatment and vaccination. The groups were well matched for age, menopausal status, tumor size, nodal status, stage of disease, estrogen receptor status, progesterone receptor status and antihormone therapy.

We claim:

1. A pharmaceutical composition comprising (i) whole autologous cancer cells, whole allogeneic cancer cells, or a combination thereof; (ii) a colony stimulating factor (CSF); (iii) an interleukin; and (iv) a pharmaceutically acceptable carrier.

2. The pharmaceutical composition of claim 1 comprising about 100,000 to about 10 million of each cancer cell type present, independently, per 100 μL of the composition.

3. The pharmaceutical composition of claim 1, comprising about 10 μg to about 100 μg of CSF per 1000 μL of the composition.

4. The pharmaceutical composition of claim 3, comprising about 15 μg to about 20 μg of CSF per 1000 μL of the composition.

5. The pharmaceutical composition of claim 1, comprising about 5,000 Units to about 50,000 Units of interleukin per 1000 μL of the composition.

6. The pharmaceutical composition of claim 5, comprising about 10,000 Units to about 30,000 Units of interleukin per 1000 μL of the composition.

7. The pharmaceutical composition of claim 1, comprising a combination of autologous cancer cells and allogeneic cancer cells.

8. The pharmaceutical composition of claim 1, comprising at least one tumor marker protein which is over-expressed by the type of tumor to be treated.

9. The pharmaceutical composition of claim 8, comprising about 1 μg to about 100 μg of each tumor marker protein per 1000 μL of composition.

10. The pharmaceutical composition of claim 8, wherein each tumor marker protein is independently carcinoembryonic antigen (CEA), CA 125 protein, CA 15-3 protein, CA 19-9 protein, prostate specific antigen (PSA), alpha feto-protein (AFP), beta human chorionic gonadotropin, ER, PgR, Her2/neu, LASA-P, PAP, PSMA, Ca 72-4, BTA, TPA, NSE, Chromogranin A, CA 50, Calcitonin, Thyroglobulin, S-100, TA-90, β-2 microglobulin, or NMP 22.

11. The pharmaceutical composition of claim 8, comprising two to five tumor marker proteins.

12. The pharmaceutical composition of claim 1, wherein the CSF comprises G-CSF, M-CSF or GM-CSF.

13. The pharmaceutical composition of claim 1, wherein the interleukin comprises IL-1, IL-2,IL-3, IL-4,IL-5, IL-6, IL-7, IL-9, IL-12, IL-13, IL-14, or IL-15.

14. The pharmaceutical composition of claim 1, comprising (i) allogeneic breast cancer cells, autologous breast cancer cells, or a combination thereof; (ii) GM-CSF; and (iii) IL-2.

15. A method for treating a cancer in a patient comprising, administering to a human patient in need thereof a therapeutically effective amount of a composition of claim 1, wherein a human patient has a solid cancerous tumor, a leukemia, or a lymphoma.

16. The method of claim 15, wherein the whole allogeneic cancer cells are obtained by either:
(i) digesting a tumor tissue to recover viable and intact tumor cells; or
(ii) collecting cells from tumor cell lines; or
(iii) separating tumor cells from blood to recover viable and intact tumor cells; and
treating the viable and intact tumor cells to yield non-viable tumor cells.

17. The method of claim 15, wherein the whole autologous cancer cells are obtained by either:
(i) digesting a tumor tissue to recover viable and intact tumor cells; or
(ii) collecting cells from tumor cell lines; or
(iii) separating tumor cells from blood to recover viable and intact tumor cells; and
treating the viable and intact tumor cells to yield non-viable tumor cells.

18. The method of claim 15, wherein each dose of the composition is administered at least one week after the previous dose.

19. The method claim 18, wherein the patient is administered 1-12 doses of the composition and the doses are administered at least one week apart.

20. The method of claim 18 wherein each of the dose comprises 300 μL to about 1000 μL of the composition.

21. A pharmaceutical composition comprising, in a liquid, pharmacologically acceptable carrier, about 100,000 to about 10 million autologous breast cancer cells, or about 100,000 to about 10 million allogeneic breast cancer cells, or a combination thereof, about 5,000 Units to about 50,000 Units of an interleukin, and about 10 μg to about 100 μg of a colony stimulating factor (CSF), per 100 μL of composition.

22. The composition of claim 21, which comprises about 500,000 to about 5 million autologous cancer cells, or about 500,000 to about 5 million allogeneic cancer cells, about 10,000 Units to about 30,000 Units of interleukin and about 15 μg to 20 μg of CSF per 100 μL of composition.

23. The composition of claim 22, wherein the interleukin is IL-2 and the CSF is GM-CSF.

24. The composition of claim 21, which further comprises at least one breast cancer marker protein.

25. The composition of claim 24, where the breast cancer marker protein comprises CEA, CA 125 or CA 15-3 or a combination thereof.

26. The composition of claim 24, wherein the or each breast cancer tumor marker protein is present in an amount of about 1 μg to about 100 μg per 100 μL of composition.

27. The composition of claim 26, which comprises, per 100 μL of composition about 500,000 to about 5 million autologous breast cancer cells, about 500,000 to about 5 million allogeneic breast cancer cells, about 10,000 Units to about 30,000 Units of IL-2, about 15 μg to 20 μg of GM-CSF, about 1 μg to about 100 μg CEA, about 100 IU to about 10,000 IU CA-125 and about 10 μg to about 500 μg CA 15-3, per 100 μL of composition.

28. A method of enhancing the immune response in a human patient who has a solid cancerous tumor, leukemia, or lymphoma to inhibit tumor growth or recurrence or to inhibit metastases formation which comprises administering to the patient a therapeutically effective amount of a composition of claim 1.

29. The method of claim 28, wherein each dose of the composition is administered at least one week after the previous dose.

30. The method claim 28, wherein the patient is administered 1-12 doses of the composition and the doses are administered at least one week apart.

31. The method of claim 29 or 30, wherein each of the dose comprises 300 μL to about 1000 μL of the composition.
